(19) United States
(12) Patent Application Publication
(10) Pub. No.: US 2008/0183252 A1
(43) Pub. Date: Jul. 31, 2008

(54) APPARATUS AND METHOD FOR TREATING CELLULITE

(76) Inventor: Roee KHEN, Haifa (IL)

Correspondence Address: Pearl Cohen Zedek Latzer, LLP, 1500 Broadway, 12th Floor, New York, NY, 10036, US

(21) Appl. No.: 11/844,274

(22) Filed: Aug. 23, 2007

Related U.S. Application Data
(60) Provisional application No. 60/836,135, filed on Sep. 5, 2006.

(51) Int. Cl. A61N 1/06; A61F 7/00

(52) U.S. Cl. 607/101; 607/115

(57) ABSTRACT

The present invention is directed to medical or aesthetic devices for treating cellulite by combining mechanical and electrical energy. The device according to embodiments of the present invention enables reducing the cellulite in the treated tissue, by using pressure on the tissue and/or by inducing heat into the treated tissue. The pressure may be provided on the tissue by a rotatable treatment member, which may be spring-loaded to exert pressure on the tissue. The heat may be induced by RF energy provided by electrodes. The RF electrodes may be combined with the rotatable treatment members.

### CROSS REFERENCE TO RELATED APPLICATIONS

This application claims the benefit of U.S. Ser. No. 60/836,135, filed on Sep. 5, 2006, which is incorporated in its entirety herein by reference.

### BACKGROUND OF THE INVENTION

1. Field of the Invention

The present invention is directed to medical or aesthetic devices for treating cellulite, and in particular, to such devices that operate using radio frequency (RF) energy. An apparatus according to embodiments of the present invention may combine mechanical and electrical energy to treat or reduce cellulite with heretofore unrealized results.

The device according to embodiments of the present invention enables reducing the cellulite in the treated tissue, by using pressure on the tissue and/or by inducing heat into the treated tissue. The pressure may be provided on the tissue by a rotatable treatment member, which may be spring-loaded to exert pressure on the tissue. The heat may be induced by RF energy provided by electrodes. The RF electrodes may be combined with the rotatable treatment members.

The device may be coupled to the treated tissue by vacuum. The device according to embodiments of the present invention may comprise means to prevent protrusion of the tissue into the device, since such protrusion may interrupt the treatment according to embodiments of the present invention.

2. Description of the Related Art

Cellulite is a subcutaneous fat tissue that protrudes into the dermis, creating an undulating dermal-subcutaneous fat junction adipose tissue, which causes dimpling of skin. As the fields of cosmetics and plastic surgery treatments develop, there is an increasing demand for treatments for destroying or reducing cellulite tissues, primarily for aesthetic reasons. One known method of removing excess adipose or fat tissue is liposuction. This is an invasive procedure in which the fat is destroyed mechanically and then extracted from under the skin using a suction device. As with any invasive surgical procedure, the procedure carries with it health risks to the patient. Moreover, liposuction is not effective for cellulite reduction. There is therefore a great need in the art for a device and a non-invasive method for efficiently treating cellulite, which is relatively easy, efficacious, and cost effective to apply.

Accordingly, there is now provided with this invention an improved apparatus and method for effectively overcoming the aforementioned difficulties and longstanding problems inherent in cellulite reduction. These problems have been solved in a simple, convenient, and highly effective way by which to treat cellulite.

### SUMMARY OF THE INVENTION

According to one aspect of the invention. An apparatus is disclosed comprising a shaft positioned in the apparatus and rotatable about an axis. At least one treatment member and a means for inducing positive pressure of said treatment member against tissue is attached to the shaft. At least one electrode is associated with the treatment member. The apparatus may further comprise a first chamber for housing the shaft, the treatment member, and the pressure inducing means and a second chamber having a vacuum duct in communication therewith adapted for having a vacuum applied thereto for coupling said apparatus to the tissue by reduced pressure.

As will be appreciated by those persons skilled in the art, a major advantage provided by the present invention is the ability to treat cellulite by including deep rotating massage, tissue lift, and squeezing preferably combined with various heating mechanisms. It is therefore an object of the present invention to improve the cellulite removal. Additional objects of the present invention will become apparent from the following description.

The method and apparatus of the present invention will be better understood by reference to the following detailed discussion of specific embodiments and the attached figures which illustrate and exemplify such embodiments.

### DESCRIPTION OF THE DRAWINGS

A specific embodiment of the present invention will be described with reference to the following drawings, wherein:

FIG. 1 is a schematic illustration of a device according to one embodiment of the present invention.

FIG. 2 is a schematic illustration from beneath of a device according to one embodiment of the present invention.

FIG. 3 is a bottom view of a schematic illustration of an embodiment of rotatable members.

### DESCRIPTION OF THE PREFERRED EMBODIMENT

The following preferred embodiment as exemplified by the drawings is illustrative of the invention and is not intended to limit the invention as encompassed by the claims of this application. An apparatus and method for treating cellulite is disclosed herein.

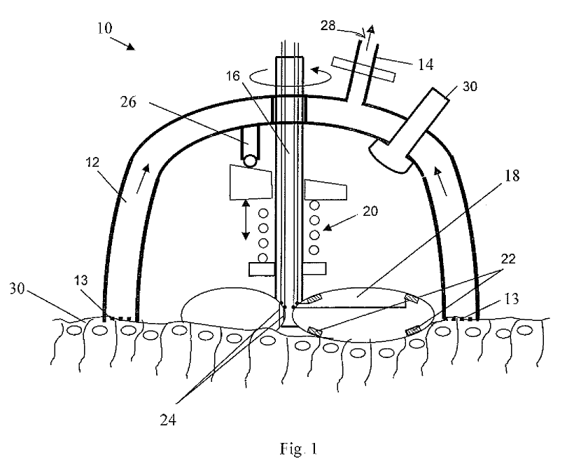

The apparatus 1, as illustrated generally in FIG. 1, is a schematic illustration of a device 10 according to one embodiment of the present invention. The device 10 may comprise a vacuum chamber 12 for delivering a vacuum 28. The vacuum may be delivered (e.g., air may be removed) via vacuum chamber 12, thereby coupling the device 10 to tissue 30. The vacuum may be delivered via vacuum chamber 12 through, for example, a vacuum duct 14. The vacuum chamber 12 may comprise a net or a wire mesh 13 at the lower edges of the vacuum chamber 12 which may be in contact with the tissue 30. The mesh 13 may prevent protrusion of portions of the tissue into the vacuum chamber.

The device may further comprise a shaft 16. In some embodiments, the vacuum may also be delivered via the shaft 16, thus further coupling the device to the tissue. In such embodiments, the shaft 16 may also comprise a mesh (not shown), functioning as described above. The shaft may be rotatable on its axis. The shaft may also be attached to treatment members 18. The treatment members 18 may provide heat to the tissue, for example, by inducing RF energy into the tissue. The treatment members 18 may include electrodes 22 which may conduct the RF energy, for example, through a layer of conductive material spread on the tissue, such as a layer of conductive gel or lotion. The conductive material may be induced onto the tissue through, for example, the shaft 16 or the treatment members 18. To prevent suction of the conductive material into the vacuum chamber when vacuum is applied, a filter (not shown) may be installed. In the embodiments in which vacuum is also delivered via the shaft, a filter may also be installed in the shaft. The RF energy may be may be provided, for example, through either the shaft or by any other conductive medium. For example, the RF energy can be delivered onto the tissue using via the electrodes 22 in all or in any of the treatment members (3 in this embodiment). Preferably, no wires should be used in the shaft. Instead, a slip ring mechanism 24 may be used. By means of electronic control, each set of electrodes 22 in each of the treatment members can be activated separately. Also, the RF level may preferably be determined by the user. The RF energy will be delivered from the shaft to the electrodes by preferably using the slip ring mechanism. Although the electrodes are generally depicted as surrounding each treatment member, the current between the electrodes should move to the tissue due to its lower resistance as compared to air and which is intensified due to the existence of the lotion or gel.

The shaft and the treatment members may induce pressure onto the tissue. The pressure induced on the tissue by the shaft and the treatment members may be regulated by a spring control 20. The pressure may be regulated manually or automatically. The spring controls 20 may adjust the pressure induced on the tissue according to the internal tension of the tissue. When the shaft 16 rotates, the treatment members 18 may rotate accordingly on the axis of the shaft, thus inducing periodic pressure on each treated point on of the tissue. The conductive material may also reduce the friction between the device and the tissue. The pressure provided by the treatment members on the tissue may be either constant or varying. For example, periodic pressure may be applied to the tissue by using a plunger 26. The plunger may be pressed against an inclined surface connected to the rotating shaft. Other axial movement actuators can also be used. In any case, the total force induced by the vacuum chamber (the opening area of 13 times the vacuum level which should be in the range of from about 080 atmospheres to about 0.95 atmospheres) should be higher than the force applied to the tissue using the loaded spring 20 so that the electrodes will not be detached from the tissue during the treatment. In this way, the operator will not have to press the device (typically attached to a hand piece to which the tip is attached) towards the tissue. This feature will enhance the ergonomic use of the tip.

The size of the treatment members should be approximately 15-30 mm and the overall diameter of the device should be about 45-90 mm.

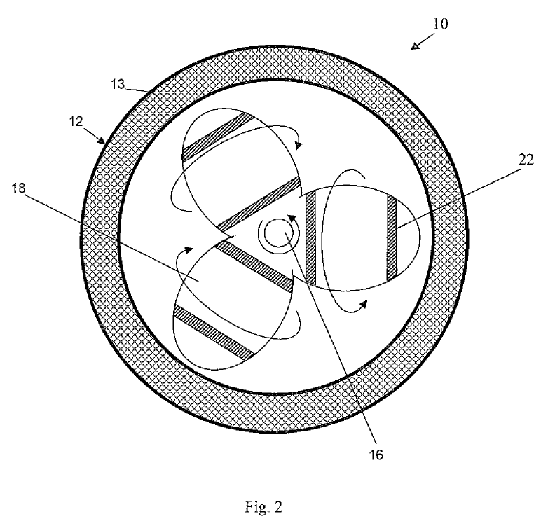

FIG. 2, which is a schematic illustration of a bottom view of the device according to one embodiment of the present invention, shows three treatment members 18. The device 10 may comprise either one treatment member or any larger number of treatment members. The treatment members may comprise one pair of electrodes 22 or any larger number of pairs of electrodes 22. For illustration purposes only, FIG. 2 shows one pair of electrodes 22 on each of the treatment members. In some embodiments of the present invention, each of treatment members may revolve about an axis parallel to the plane of the tissue (shown in FIG. 1), alternatively or in addition to the rotation about the shaft. The kind of revolution of the treatment members depicted in FIG. 2 may also reduce friction between the device and the tissue. The revolution of the treatment members about an axis parallel to the plane of the tissue can be either made by placing small motors inside the treatment members synchronized with the rotation of the shaft or passively so that only a bearing is connecting each of members is to the shaft 16. Generally, two motions of the members 18 may be possible with respect to the tissue, i.e. sliding or rotation. Sliding may disturb the patient so rotation is often the preferred motion. In such an embodiment, the additional function from the protrusion extending from the treatment members will enforce rotation of the treatment members and prevent them from slipping.

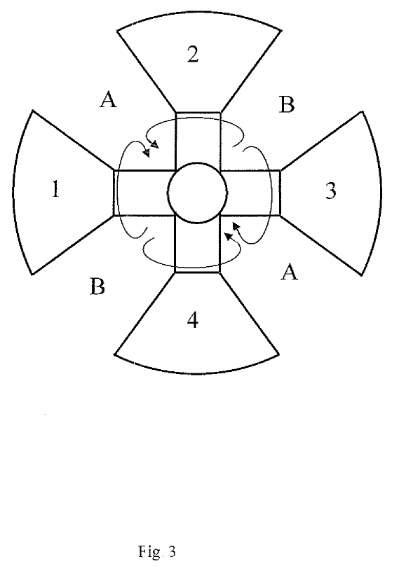

Alternative shapes of the treatment members may also be used and may be found advantageous. Further, the direction of rotation of the treatment members may be determined by the user. In the case where all the members are rotating in the same direction (which is directly related to the direction of rotation of the main shaft 16) the tip rotationally massages the tissue. An alternative is to stop the rotation of shaft and to let each member 18 rotate independently of the other members. In one embodiment, by choosing the direction of rotation shown in FIG. 3, the tissue may be pulled into the space between the members which are rotating in opposite directions. (Such a configuration would require an even number of members so that each two of the members form a treatment cell.)

Referring to FIG. 3 which views the treatment members from the tissue side, the areas marked ‘A’ and ‘B’ form treatment cells. As the direction of the members may be predefined by the user, it can be seen that depending upon the specific direction of the members, as shown in FIG. 3 where the rotation directions are marked with arrows, each set of members rotate against each other. In these treatment cells, the tissue is drawn into the space A between treatment members 1 and 2 and between members 3 and 4. At the same time, the tissue in the areas marked ‘B’ are stretched to enable the tissue protrusion into at areas A.

Heat may be added by a wide variety of methods including convection, radiative, or induction. For example, heating coils, or a wide array of electromagnetic radiation may be used to heat the treated tissue. Such electromagnetic radiation 30 may be applied to the treatment chamber and may be in the form of monochromatic light such as lasers or as white light. Additionally, infra-red light may be used to heat the treatment chamber and to heat the treated tissue as shown in FIG. 1. The electromagnetic radiation may be employed continuously, intermittently, or in pulses in accordance with a predetermined frequency.

Although the particular embodiments shown and described above will prove to be useful in many applications in the cellulite treatment art to which the present invention pertains, further modifications of the present invention will occur to persons skilled in the art. All such modifications are deemed to be within the scope and spirit of the present invention as defined by the appended claims.

What is claimed is:

1. An apparatus, comprising:
a shaft positioned in the apparatus and rotatable about an axis;
at least one treatment member attached to said shaft;
means for inducing positive pressure of said treatment member against tissue when placed thereon; and
at least one electrode associated with said at least one treatment member.

2. The apparatus of claim 1, further comprising:
a. a first chamber for housing said shaft, said at least one treatment member, and said pressure inducing means; and
b. a second chamber having a vacuum duct in communication therewith adapted for having a vacuum applied thereto for coupling said apparatus to said tissue by reduced pressure.

3. The apparatus of claim 2, wherein said second chamber further comprises means for preventing protrusion of said tissue into said second chamber.

4. The apparatus of claim 1, wherein the electrode is a radio frequency electrode.

5. The apparatus of claim 1, further comprising a source of heat.

6. The apparatus of claim 5, wherein said source of heat is a conductive element.

7. The apparatus of claim 6, wherein the source of heat is electromagnetic radiation.

8. The apparatus of claim 7, wherein the electromagnetic radiation is applied in pulses.

9. The apparatus of claim 2, wherein the electrode is a radio frequency electrode.

10. The apparatus of claim 9, further comprising a source of heat positioned within said first chamber.

11. The apparatus of claim 10, wherein the source of heat is a conductive element.

12. The apparatus of claim 10, wherein the source of heat is electromagnetic radiation.

13. The apparatus of claim 12, wherein the electromagnetic radiation is applied in pulses.
